(19) United States
(12) Patent Application Publication
(10) Pub. No.: US 2016/0145686 A1
(43) Pub. Date: May 26, 2016

(54) BIOMARKER FOR SENESCENCE AND USE THEREOF

(71) Applicant: Samsung Electronics Co., Ltd., Suwon-si (KR)

(72) Inventors: Myoungsoon Kim, Anyang-si (KR); Youngsam Lee, Seoul (KR); Yongsub Kim, Suwon-si (KR); Hyuntae Kang, Seoul (KR)

(21) Appl. No.: 14/821,104

(22) Filed: Aug. 7, 2015

(30) Foreign Application Priority Data
Nov. 26, 2014 (KR) 10-2014-0166631

(51) Int. Cl. C12Q 1/68

(52) U.S. Cl. CPC C12Q 1/6883; C12Q 2600/158; C12Q 2600/136

(57) ABSTRACT

A biomarker for diagnosing senescence, a composition and a kit for diagnosing a senescence level to detect the same, a method of diagnosing a senescence level in a cell or subject, and a method of screening a senescence inhibitor.

### RELATED APPLICATION

This application claims the benefit of Korean Patent Application No. 10-2014-0166631, filed on Nov. 26, 2014, in the Korean Intellectual Property Office, the entire disclosure of which is hereby incorporated by reference.

### INCORPORATION BY REFERENCE OF ELECTRONICALLY SUBMITTED MATERIALS

Incorporated by reference in its entirety herein is a computer-readable nucleotide/amino acid sequence listing submitted herewith and identified as follows: One 4,138 byte ASCII (Text) file named “720624_ST25.TXT” created Aug. 7, 2015.

### BACKGROUND

1. Field

The present disclosure relates to biomarkers for diagnosing senescence and methods using the same.

2. Description of the Related Art

Senescence or aging is a disruptive phenomenon that occurs over time. While some biological activities generally deteriorate with aging in humans, activities of some enzymes or secretion of some hormones may increase. Cellular senescence may be defined as a permanent halt of cellular division. Replicative senescence or cellular senescence has been observed as an aging model in the cell level. When cells are continuously cultured, cells divide a number of times, but aged cells can no longer divide. In fact, senescent cells are resistant against programmed cell death, and some senescent cells are maintained in a non-dividing state for several years.

Although lipofuscin and lipid peroxide are widely known as biomarkers for diagnosing senescence, there is still a need to develop and identify a biomarker for diagnosing senescence with high accuracy. Although methods of measuring β-galactosidase have been used to measure a senescence level, results cannot be reliably quantified.

Thus, there is a need to develop a biomarker for diagnosing senescence and a method of quantitatively measuring a senescence level by using the same.

### SUMMARY

Provided is a composition and kit for diagnosing a senescence level comprising a nucleic acid (e.g., cDNA) consisting of SEQ ID NO: 1 a sequence complementary to SEQ ID NO: 1, or a polynucleotide fragment thereof.

Provided is a method of diagnosing a senescence level of a cell or a subject, the method comprising: separating ribonucleic acids (RNA) from a biological sample of a cell or a subject; generating complementary DNAs (cDNA) from the separated RNAs; measuring the amount of SEQ ID NO: 1 or fragment thereof comprising about 4 nucleotides to about 100 nucleotides that is present in the cDNAs; and comparing the measured amount of SEQ ID NO: 1 or the fragment thereof with the amount of SEQ ID NO: 1 or the fragment thereof obtained from a control group sample to determine a senescence level of the cell or the subject.

Provided is a method of screening for a senescence inhibitor, the method comprising: incubating cells with a test compound; separating RNAs from the cells; generating cDNAs from the separated RNAs; measuring the amount of SEQ ID NO: 1 or fragment thereof comprising about 2 nucleotides to about 100 nucleotides in the cDNAs, that is present in the cDNAs; and determining that the test compound is a senescence inhibitor when an amount of the SEQ ID NO: 1 or the fragment thereof in the cells incubated with the test compound decreases in comparison with a control group

Related compositions and methods are provided herein.

### BRIEF DESCRIPTION OF THE DRAWINGS

These and/or other aspects will become apparent and more readily appreciated from the following description of the exemplary embodiments, taken in conjunction with the accompanying drawings in which:

FIGS. 1A and 1B are graphs illustrating RT-qPCR results and microarray analysis results of Linc_WARC1 present in young and old cells (Dashed line: primer set 1 and Dashed line: primer set 2), respectively;

FIGS. 2A to 2F are graphs illustrating RT-qPCR results of Linc_WARC1 in young cells and senescent cells of HDF cells (M11), HDF cells (M4), myoblast cells, HMECs, myoblast cells (osteoblast), and IMR-90 cells (Young: young cell and Old: senescent cell), respectively;

FIGS. 3A to 3D are graphs illustrating RT-qPCR results of Linc_WARC1 in young cells, middle-aged cells, and senescent cells of HDF cells (M11), HDF cells (M4), myoblast cells, and HMECs (Young: young cell, Middle: middle-aged cell, and Old: senescent cell);

FIG. 4 is a diagram illustrating a location of Linc_WARC1 at position 12.3 on p-arm of human chromosome 16; and

FIG. 5 is a graph illustrating RT-qPCR results of Linc_WARC1 in young cells and senescent cells of dermal fibroblast cells of a patient with Hutchinson-Gilford Progeria Syndrome (HGPS) (Young: young cell and Old: senescent cell).

### DETAILED DESCRIPTION

Reference will now be made in detail to exemplary embodiments, examples of which are illustrated in the accompanying drawings, wherein like reference numerals refer to like elements throughout. In this regard, the present exemplary embodiments may have different forms and should not be construed as being limited to the descriptions set forth herein. Accordingly, the exemplary embodiments are merely described below, by referring to the figures, to explain aspects. As used herein, the term “and/or” includes any and all combinations of one or more of the associated listed items. Expressions such as “at least one of,” when preceding a list of elements, modify the entire list of elements and do not modify the individual elements of the list. Additional aspects will be set forth in part in the description which follows and, in part, will be apparent from the description, or may be learned by practice of the presented exemplary embodiments.

According to an aspect of an exemplary embodiment, provided is a composition for diagnosing a senescence level according to an exemplary embodiment includes a nucleic acid consisting of a nucleotide sequence of SEQ ID NO: 1 or a polynucleotide complementary to of SEQ ID NO: 1, or a fragment thereof.

The term ‘senescence’, may be defined as deteriorative changes occurring over time. Used herein, the phrase “cell senescence” includes the reduced ability of a cell to proliferate compared with reference cells (i.e., a non-senescent cell of the same type), an increase in accumulation of lipofuscin, an increase in the activity of β-galactosidase, an increase in mitochondrial reactive oxygen species, a decrease in mitochondrial membrane potentials, and/or an increase in duration of G0 and/or G1 phase of the cells compared to a reference cell. The phrase “young cell” refers to a cell with enhanced ability to proliferate, a decrease in lipofuscin accumulation, a decrease in the activity of β-galactosidase, a decrease in mitochondrial reactive oxygen species, an increase in mitochondrial membrane potentials, and a decrease in duration of G0 and/or G1 phase of the cell occurs, when compared to a reference cell (i.e., a senescent cell of the same type). For example, when doubling time of a first set of cells is greater than twice, three times, four times, five times, six times, seven times, nine times, ten times, fifty times, or hundred times than that the doubling time of a second set of cells of the same type that have undergone the same number of cycles of passaging or cell subculturing as the first set of cells, the second set of cells may be referred to as senescent cells. In case of humans, cells from humans over about 30 years old, over about 40 years old, over about 50 years old, over about 60 years old, over about 70 years old, over about 80 years old, over about 90 years old, or over about 100 years old may be regarded as senescent cells.

The “diagnosing of the senescence level” or “determining a senescence level” may be used for diagnosing a senescence-associated disease by analyzing one or more of the factors described above and comparing said one or more factors to a reference cell, providing information related to the senescence level, or quantifying a target nucleic acid that is a biomarker for cellular senescence. Examples of the senescence-associated disease may include progeria, cognitive disease (including Alzheimer's disease, Parkinson's disease, dementia, or a combination thereof), stroke, diabetes, arthritis, arteriosclerosis, heart disease, hair loss, wrinkles, and osteoporosis. The information related to the senescence level may be information related to biological age. The term “biological age”, also called physiological age, is a time-based approach to describe growth, maturation, or aging of a living body (e.g., a cell). Although age may be measurable using a calendar criteria in years, months, and days, i.e., may be expressed as a chronological age, growth, maturation, or aging do not progress in the same manner in all subjects, and thus the biological age of a living body determined from senescence level provides information that is more relevant for some uses than chronological age. The biological age may be quantified by measuring 353 epigenetic markers on the DNA. The 353 markers measure DNA methylation of CpG dinucleotides.

The nucleic acid consisting of a nucleotide sequence of SEQ ID NO: 1, sequence complementary to SEQ ID NO: 1, or fragment thereof, may be a noncoding ribonucleic acid (ncRNA). The ncRNA is a functional RNA molecule which is not translated into a protein. The ncRNA may be small nucleolar RNA (snoRNA), microRNA, small interfering RNA (siRNA), small nuclear RNA (snRNA), extracellular RNA (exRNA), Piwi-interacting RNA (piRNA), or long ncRNA (IncRNA). The IncRNA is a transcript, which is not translated into a protein or has a low protein-coding potential, and has a length of about 50 nucleotides (nt) or greater, about 100 nt or greater, about 200 nt or greater, or about 500 nt or greater. The nucleic acid consisting of a nucleotide sequence of SEQ ID NO: 1, sequence complementary to SEQ ID NO: 1, or fragment thereof may be a nucleic acid transcribed from a nucleic acid located at p-arm of human chromosome 16. The nucleic acid consisting of a nucleotide sequence of SEQ ID NO: 1, sequence complementary to SEQ ID NO: 1, or fragment thereof may be a nucleic acid transcribed from a nucleic acid located between a ERI2 gene (ERI1 Exoribonuclease Family Member 2) of human chromosome 16 and a DCUN1D3 gene (DCN1, Defective In Cullin Neddylation 1, Domain Containing 3) gene. The nucleic acid consisting of the nucleotide sequence of SEQ ID NO: 1, sequence complementary to SEQ ID NO: 1, or fragment thereof, may have a nucleotide sequence of GenBank Accession No. AK027199.

The fragment may be a polynucleotide having a nucleotide sequence of two or more continuous nucleotides among nucleotides of the nucleic acid consisting of a nucleotide sequence of SEQ ID NO: 1. The fragment may have a length of about 2 nucleotides (nt) to about 2000 nt, about 3 nt to about 1500 nt, about 4 nt to about 1000 nt, about 4 nt to about 500 nt, about 4 nt to about 100 nt, about 5 nt to about 500 nt, about 6 nt to about 200 nt, about 7 nt to about 100 nt, about 8 nt to about 50 nt, about 9 nt to about 40 nt, or about 10 nt to about 30 nt.

The polynucleotide may be a primer or probe. The polynucleotide may have a length of about 2 nt to about 100 nt, about 3 nt to about 90 nt, about 4 nt to about 80 nt, about 5 nt to about 70 nt, about 6 nt to about 60 nt, about 7 nt to about 50 nt, about 8 nt to about 40 nt, about 9 nt to about 30 nt, or about 10 nt to about 30 nt. As used herein the term “primer” may be a nucleic acid fragment serving as a starting point of DNA synthesis. The primer may be a forward primer or a reverse primer. As used herein the term “probe” refers to an oligomer including DNA and/or RNA which may be hybridized with a specific nucleotide sequence. The probe may be labeled with a detectable label. For example, the detectable label may be a fluorescent label.

The composition may further include a substance used to detect the nucleic acid consisting of a nucleotide sequence of SEQ ID NO: 1, sequence complementary to SEQ ID NO: 1, or fragment thereof. For example, the composition may further include a polymerase, a buffer solution, dNTP, a detection reagent, or any combination thereof.

According to an aspect of another embodiment, provided is a kit for diagnosing a senescence level, wherein the kit comprises a nucleic acid consisting of a nucleotide sequence of SEQ ID NO: 1, sequence complementary to SEQ ID NO: 1, or fragment thereof, and a detection reagent.

The nucleic acid consisting of a nucleotide sequence of SEQ ID NO: 1, sequence complementary to SEQ ID NO: 1, or fragment thereof the senescence, and the diagnosing of the senescence level are as described above.

The detection reagent may be a substance used to detect the nucleic acid consisting of a nucleotide sequence of SEQ ID NO: 1, sequence complementary to SEQ ID NO: 1, or fragment thereof. For example, the detection reagent may be an enzyme or a buffer solution.

According to an aspect of another exemplary embodiment, provided is a method of diagnosing or determining a senescence level of a cell or a subject comprising separating nucleic acids from a biological sample, and quantifying a nucleic acid consisting of a nucleotide sequence of SEQ ID NO: 1, sequence complementary to SEQ ID NO: 1, or fragment thereof in the separated nucleic acids, such as by generating cDNA of the nucleic acid and quantifying the cDNA.

As used herein the phrase “target nucleic acid” refers to a nucleic acid to be quantified and/or analyzed in the biological sample, such as a nucleic acid comprising or consisting of SEQ ID NO: 1, sequence complementary to SEQ ID NO: 1, or fragment thereof, or a cDNA thereof.

The nucleic acid consisting of a nucleotide sequence of SEQ ID NO: 1, sequence complementary to SEQ ID NO: 1, or fragment thereof, the polynucleotide, the senescence, and the diagnosing of the senescence level are as described above.

The biological sample may be a sample separated from a subject having a senescence-associated disease or a risk of having a senescence-associated disease, or a cultured cell. The biological sample may be blood, plasma, serum, tissues, urine, mucus, saliva, tears, sputum, spinal fluid, pleural fluid, nipple aspirate, lymph fluid, respiratory tract fluid, serous fluid, urogenital fluid, breast milk, lymph secretion, sperm, cerebrospinal fluid, secretion in organs, abdominal fluid, fluid from cystic tumor, amniotic fluid, or any combination thereof.

The subject may be a mammal. The mammal may be human, dog, cat, sheep, pig, mouse, rabbit, hamster, rat, or guinea pig.

The cell may be a cell separated from a subject or a cultured cell. The cell may be a nerve cell, an immune cell, an epithelial cell, a germ cell, a muscle cell, or a cancer cell. The cell may be a cell line or a primarily cultured cell.

The nucleic acid may be ribonucleic acid (RNA). The RNA may be a transcript produced from a genome.

Any method commonly used in the art may be used to separate nucleic acids from the biological sample.

The quantifying of the polynucleotide (i.e., the target nucleic acid) may be performed using a method commonly used in the art. For example, the method may be performed by reverse transcription-polymerase chain reaction (RT-PCR), microarray analysis, serial analysis of gene expression (SAGE), Northern blotting, or any combination thereof. Quantifying may involve generating cDNA and quantifying the cDNA.

The method may further include determining that the biological sample is a sample having an increased senescence level when an amount the target nucleic acid increases (i.e., is greater than) by about 1.5 times to about 10 times, for example, about 1.5 times to about 8 times, about 1.5 times to about 6 times, or about 1.5 times to about 4 times the level of the target nucleic acid of a control group. The method may further include determining that the biological sample is a sample having a decreased senescence level when the target nucleic acid decreases (i.e., is less than) by about 0.1 times to about 0.9 times, for example, about 0.2 times to about 0.8 times, about 0.3 times to about 0.7 times, or about 0.4 times to about 0.6 times that of a control group. The control group (i.e., a normal control group), may be a sample separated from a subject of about 30 years old or less, a subject or a cell not having a senescence-associated disease (e.g., progeria), or a subject or cell not having a risk of having a senescence-associated disease.

According to an aspect of another exemplary embodiment, provided is a method of screening a senescence inhibitor and a test compound, comprising separating nucleic acids from the cells, quantifying a target nucleic acid that is complementary to SEQ ID NO: 1 or a fragment thereof contained in the separated nucleic acids, and determining that the test compound is a senescence inhibitor when an amount of the target nucleic acid decreases in comparison with a control group.

The cells, the separating of nucleic acids, the nucleic acid consisting of a nucleotide sequence of SEQ ID NO: 1 a polynucleotide fragment thereof, or a nucleic acid that is complementary to a fragment of SEQ ID NO: 1, the fragment, the polynucleotide, the quantifying of the polynucleotide, and the senescence are as described above.

The senescence inhibitor may be a substance capable of reducing the senescence level.

The test compound may be chemicals, proteins, nucleic acids, lipids, or any combination thereof.

The incubating may be performed in vitro.

The separating of nucleic acids from the cells may be performed using a method commonly used in the art.

The control group may be a negative control group. The negative control group may be cells of the same type (e.g., from the same sample or subject) which are not incubated with the test compound.

The senescence inhibitor may be a therapeutic agent or an agent relieving symptoms of progeria, cognitive disease, stroke, diabetes, arthritis, arteriosclerosis, heart disease, hair loss, wrinkles, or osteoporosis.

When the amount of the target nucleic acid increases after incubation with a test compound in comparison with the control group, the test compound may be determined as a candidate senescence inhibitor. The senescence inhibitor may be an anticancer agent.

According to the biomarker for diagnosing senescence, the composition and kit for diagnosing a senescence level to detect senescence, the method of diagnosing the senescence level in a cell or subject, and the method of screening the senescence inhibitor, the senescence level may be quantitatively measured with high accuracy. In addition, they may be used to determine the senescence level and screen the senescence inhibitor.

Hereinafter, one or more embodiments of the inventive concept will be described in detail with reference to the following examples. These examples are not intended to limit the purpose and scope of the one or more embodiments of the inventive concept.

### Example 1

### Identification of Senescence-Associated Biomarker of Long Non-Coding RNA (Lnc RNA)

(1) Screening of Senescence-Associated Lnc RNA

In order to screen a senescence-associated biomarker, human dermal fibroblast (HDF) M11 cells obtained from a 11-year-old boy were cultured in Dulbecco's Modified Eagle's Media (DMEM) including high-concentration glucose, glutamine, and pyruvate and supplemented with 10% (v/v) fetal bovine serum (FBS) and 1× penicillin/streptomycin in a 5% CO₂ incubator at 37° C., thereby obtaining young cells and senescent cells. HDF M11 cells (passage number 14) having a cell doubling time of about 1 day were used as the young cells, and HDF M11 cells (passage number 38) having a cell doubling time of about 14 days or more were used as the senescent cells.

Meanwhile, the senescent cells were restored to young cells by culturing the senescent cells in an extracellular matrix (ECM) obtained from the young cells. The ECM of the young cells was obtained according to a method disclosed in Aging Cell (2011), vol. 10: pp. 148-157. The senescent cells were seeded on the ECM obtained from the young cells and cultured for 2 days, 8 days, 16 days, or 24 days at 37° C. in 5% CO₂ atmosphere. After the ECM of the young cells was added to the senescent cells, and the cultured cells for 2 days, 8 days, 16 days, or 24 days were labeled with R2d, R8d, R16d, and R24d, respectively.

RNAs were obtained from collected cells by adding a TRIZOL® (Life technologies) to the cells, lysing the cells, adding chloroform thereto, and homogenizing the resultant. The resultant was centrifuged to separate RNAs from a supernatant. The same amount of isopropanol was added to the separated supernatant, and the mixture was centrifuged to obtain precipitated RNAs.

Complementary DNAs (cDNA) with fluorescent labels were prepared by labeling 1 μg of the obtained RNAs with fluorescent dyes by using an amino-allyl cDNA Labeling kit (Ambion). RNA obtained from the young cells was labeled with Cy3, and RNA obtained from the senescent cell was labeled with Cy5. The labeled RNAs were added to a human genome 8×60 k array (Agilent Technologies) and incubated at about 55° C. for about 16 hours to hybridize the RNAs. The human genome 8×60 k array is a microarray in which 7,419 large intergenic non-coding RNAs (lincRNAs) and 2,644 non-coding RNAs (ncRNAs) are integrated. Images were analyzed using ImaGene 4.2 (Biodiscovery) and MAAS (Gaiagene) software, and genes exhibiting statistically significant changes in expression levels were primarily selected by SAM analysis. Then, expression patterns were classified into A to F types by using a Cluster program.

As a result of the microarray analysis, a difference of more than 1.5 times was observed in expression levels between the young cells and the senescent cells. 35 types of LincRNAs and 37 types of noncoding RNAs were primarily selected (p<0.01). Among the primarily selected RNAs, 15 types of LincRNAs and 10 types of noncoding RNAs, expression of which decreased in R24d cells, which are the restored young cells from the senescent cells, were secondarily selected. The secondarily selected RNAs included 8 type A RNAs, 6 type B RNAs, 3 type C RNAs, 4 type D RNAs, 3 type E RNAs, and one type F RNA. The types A and B RNAs are down-regulated transcripts since amounts of transcripts of the senescent cells were less than that of the young cells. The types C to F RNAs are up-regulated transcripts since amounts of transcripts of the senescent cells were greater than that of the young cells.

(2) Verification Through RT-qPCR

Reverse transcription-quantitative polymerase chain reaction (RT-qPCR) was performed to verify the transcripts selected according to Example 1-(1).

25 μl of a reverse transcription buffer solution (including 50 mM Tris-HCl (pH 8.3), 75 mM KCl, 3 mM MgCl2, 0.1 M DTT, 10 mM dNTP, and 40 unit/μl RNase inhibitor), 0.5 μg/μl of oligo-dT16 primer, and 200 units of a SUPERSCRIPT® II reverse transcriptase (GiboBRL) were added to 1 pg of the RNAs obtained according to Example 1-(1), and the mixture was incubated at 42° C. for 1 hour. 2.5 μl of the reaction mixture was added to 50 μl of a PCR buffer solution (including 0.04 unit of an AMPLITAQ® DNA polymerase (Life Technologies), 50 mM Tris-HCl (pH 8.3), 0.25 mg/ml bovine serum albumin (BSA), 3 mM MgCl2, 0.25 mM dNTPs, and SYBR® Green I at 1/50,000 dilution (Life Technologies)), and 10 μM of forward primers and reverse primers of the respective transcripts selected according to Example 1-(1) were added thereto to prepare a reaction mixture. The reaction mixture was incubated at 94° C. for 30 seconds, at 53° C. for 30 seconds, at 72° C. for 1 minute, which is regarded as one cycle, and the incubation was repeated 30 cycles. A relative mRNA level was determined by measuring a fluorescence change of SYBR® Green I using ICycler software.

The amounts of the quantified transcript were normalized with respect to an amount of GAPDH, and fold-changes with respect to the transcripts of the senescent cells (fold-change/old) was calculated. Transcripts having similar patterns between the results of the calculated RT-qPCR and the results of the microarray analysis according to Example 1-(1) were selected and referred to as Linc_WARC1 (SEQ ID NO: 1). The Linc_WARC1 has a length of 2,118 nt. Primer sets used for amplification of Linc_WARC1 are as follows.

Primer set 1:
Forward primer:
(SEQ ID NO: 2)
5′-TTAAGCACAGACGGAGCTGG-3′
and
Reverse primer:
(SEQ ID NO: 3)
5′-TGGGCATCATGTCCTCCCTA-3′
Primer set 2:
Forward primer:
(SEQ ID NO: 4)
5′-CACAAAGGTGGAGGGTCACA-3′
and
Reverse primer:
(SEQ ID NO: 5)
5′-TGGGGTTTTTCATCCCCCTG-3′

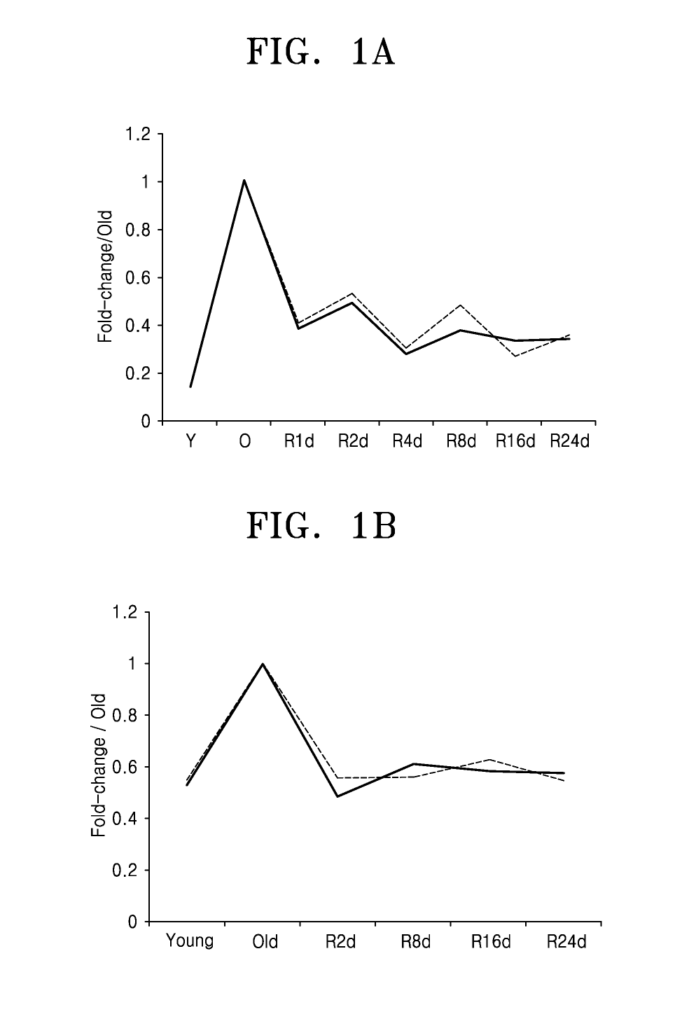

FIGS. 1A and 1B are graphs illustrating the results of the RT-qPCR and the results of microarray analysis according to Example 1-(1) (Y: young cells and O: senescent cells, R2d, R8d, R16d, and R24d; senescent cells cultured with ECM of young cells for 2 days, 8 days, 16 days, and 24 days, respectively, Dashed line (FIGS. 1A and 1B): primer set 1, Dashed line (FIGS. 1A and 1B): primer set 2). In FIGS. 1A and 1B, although dashed lines and solid lines indicate results of two probes, the probes target the same transcript.

(3) Identification of Expression of Linc_WARC1 in Various Cells

In order to identify whether the selected Linc_WARC1 is a marker available for general use in senescent cells, expression levels of Linc_WARC1 were identified between the young cells and the senescent cells in various cells.

HDF cells obtained from an 11-year-old boy (′HDF (M11)′), HDF cells obtained from a 4-year-old boy (′HDF (M4)′), myoblast cells (Lonza, Cat. No. cc-2580), human mammary epithelial cells (HMECs), osteoblast cells (SCIENCELL™, Cat. No. 4610), and human lung fibroblast (IMR-90) cells (ATCC®, CCL-186™) were prepared. As described above with reference to Example 1-(1), HDF and IMR-90 cells were cultured in DMEM (HYCLONE™) supplemented with 10% (v/v) FBS, myoblast cells were cultured in SKGM™-2 (Lonza), HMECs were cultured in MEGM™ (Lonza), and osteoblast cells were cultured in ObM (SCIENCELL™) in 5% CO₂ atmosphere at 37° C. to prepare young cells and senescent cells.

Passage numbers and doubling times of the prepared cells are shown in Table 1 below.

TABLE 1

| | Young cell | | Senescent cell | |
|---|---|---|---|---|
| | Passage | | Passage | |
| Cell | number | Doubling time | number | Doubling time |
| HDF (M11) | 14 | about 1 day | 38 | >about 14 days |
| HDF (M4) | 8 | about 1 day | 30 | about 14 days |
| Myoblast cell | 2 | about 2 days | 10 | about 7 days |
| HMEC | 4 | about 2 days | 13 | about 14 days |
| Osteoblast cell | 1 | about 2 days | 10 | about 1 month |
| IMR-90 | 3 | about 2 days | 15 | about 14 days |

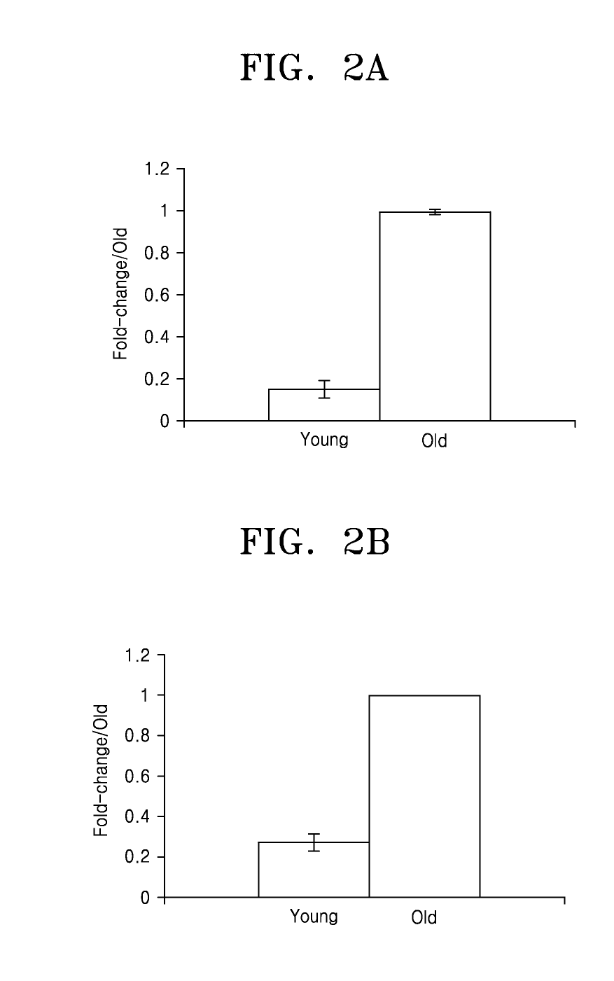

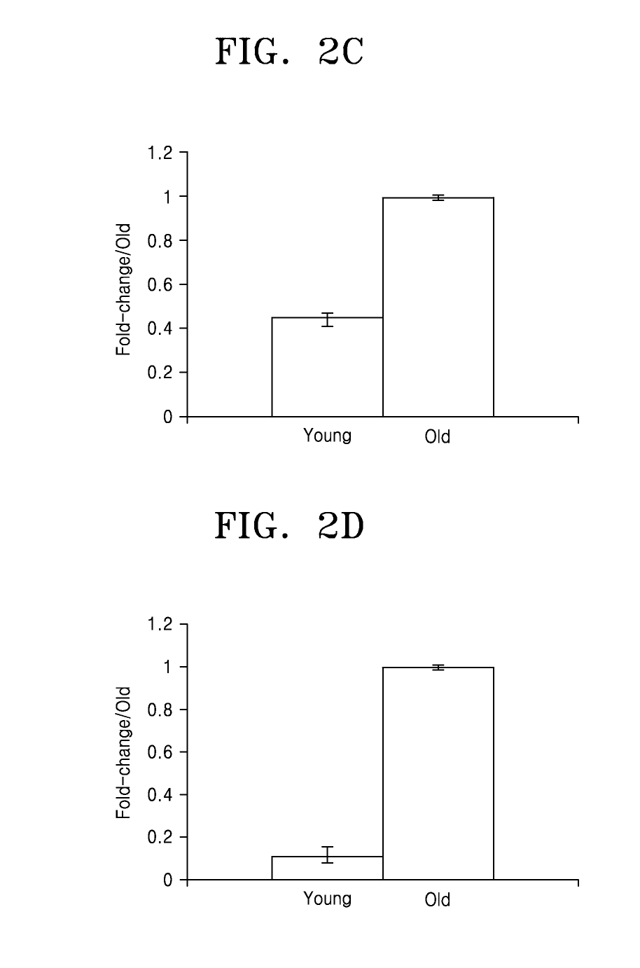

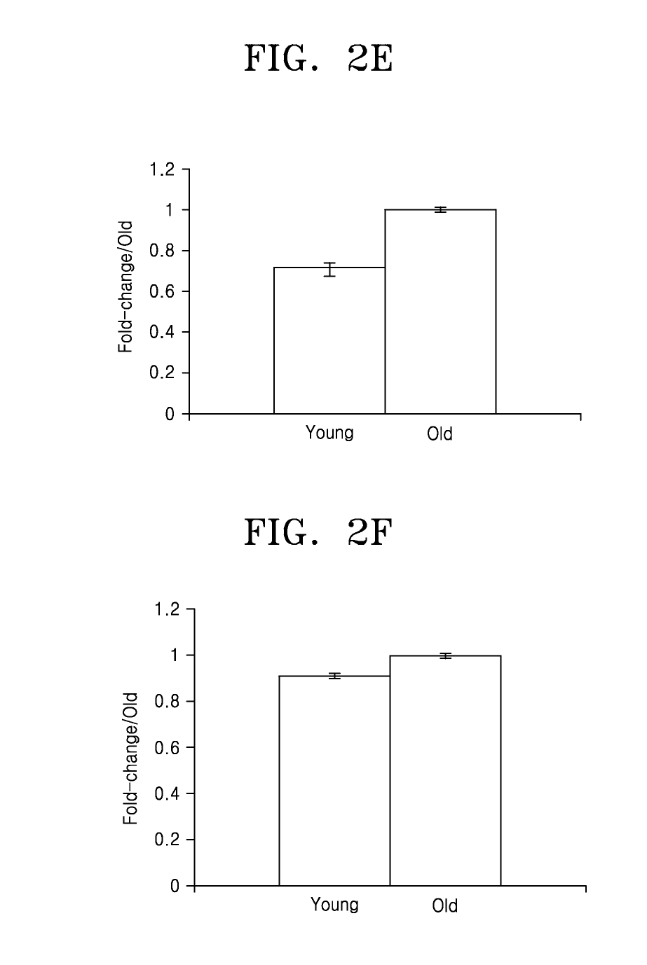

RNAs were obtained from the cultured cells according to the method described above with reference to Example 1-(1), and RT-qPCR was performed according to the method described above with reference to Example 1-(2). The amounts of the quantified transcripts were normalized with respect to an amount of GAPDH, and fold-changes with respect to the transcripts of the senescent cells (fold-change/old) were calculated. The results are shown in FIGS. 2A to 2F (Young: young cells and Old: senescent cells).

As illustrated in FIGS. 2A to 2F, expression levels of Linc_WARC1 increased in the senescent cells of HDF cells (M11), HDF cells (M4), myoblast cells, osteoblast cells, and IMR-90 cells in comparison with the young cells. Thus, it was confirmed that Linc_WARC1 is a biomarker for senescence available for general use.

(4) Analysis of Linc_WARC1 Over Time

It was identified whether expression levels of Linc_WARC1 change over time.

The HDF cells (M11), HDF cells (M4), myoblast cells, and HMECs prepared according to Example 1-(3) were cultured and classified into young cells, middle cells, and senescent cells. Passage numbers and doubling times of the young cells, middle cells, and senescent cells are shown in Table 2 below.

TABLE 2

| | Young cell | | Middle cell | | Senescent cell | |
|---|---|---|---|---|---|---|
| | Passage | Doubling | Passage | Doubling | Passage | Doubling |
| Cell | number | time | number | time | number | time |
| HDF | 14 | about 1 | 28 | about 7 | 38 | >about 14 |
| (M11) | | day | | days | | days |
| HDF | 8 | about 1 | 21 | about 6 | 30 | about 14 |
| (M4) | | day | | days | | days |
| Myo- | 2 | about 2 | 6 | about 6 | 10 | about 7 |
| blast | | days | | days | | days |
| cell | | | | | | |
| HMEC | 4 | about 2 | 7 | about 5 | 13 | about 14 |
| | | days | | days | | days |

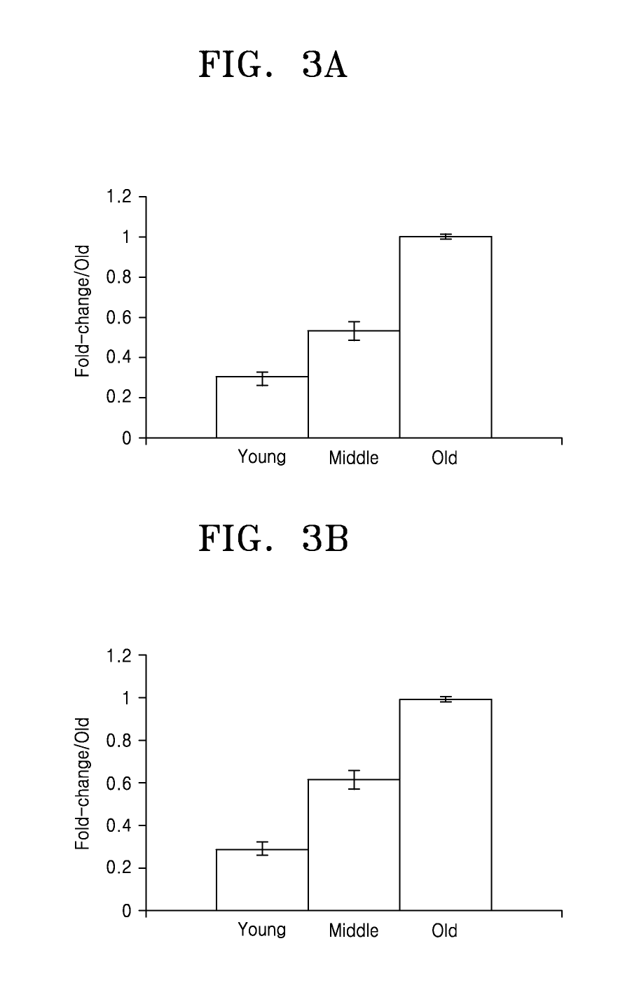

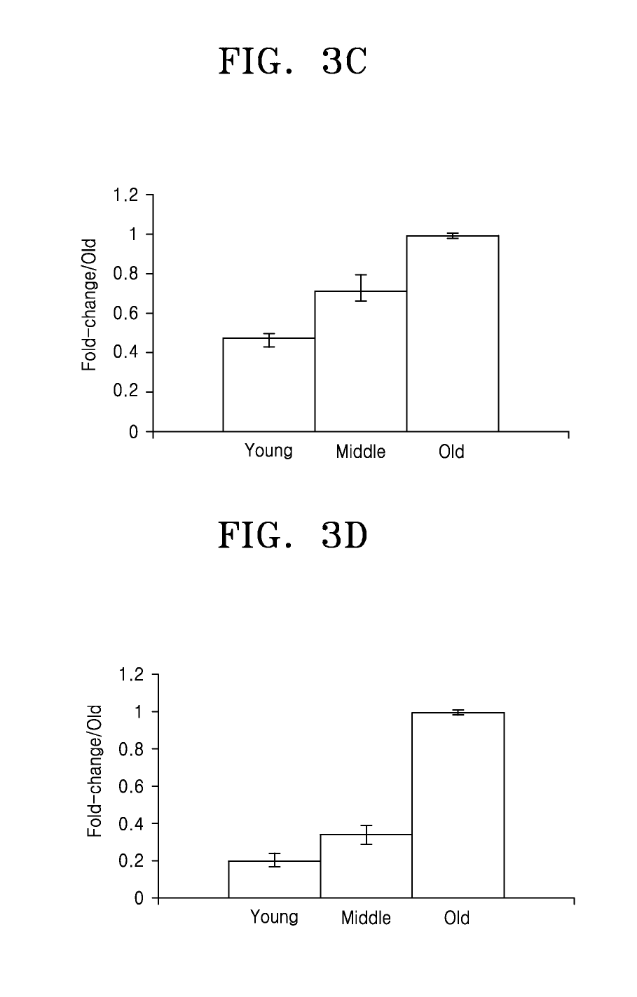

RNAs were obtained from the cultured cells according to the method described above with reference to Example 1-(1), and RT-qPCR was performed according to the method described above with reference to Example 1-(2). The amounts of the quantified transcripts were normalized with respect to an amount of GAPDH, and fold-changes with respect to the transcripts of the senescent cells (fold-change/old) were calculated. The results are shown in FIGS. 3A to 3D (Young: young cells, Middle: middle cell, and Old: senescent cells).

As illustrated in FIGS. 3A to 3D, expression levels of Linc_WARC1 increased over time in the HDF cells (M11), HDF cells (M4), myoblast cells, and HMECs. Thus, it was verified that Linc_WARC1 is a biomarker for senescence for general use.

(5) Identification of Linc_WARC1

A location of Linc_WARC1 within a chromosome was identified by inputting a microarray probe sequence of a gene to the UCSC Genome Bioinformatics website (genome.ucsc.edu/) or the NCBI website (www.ncbi.nlm.nih.gov).

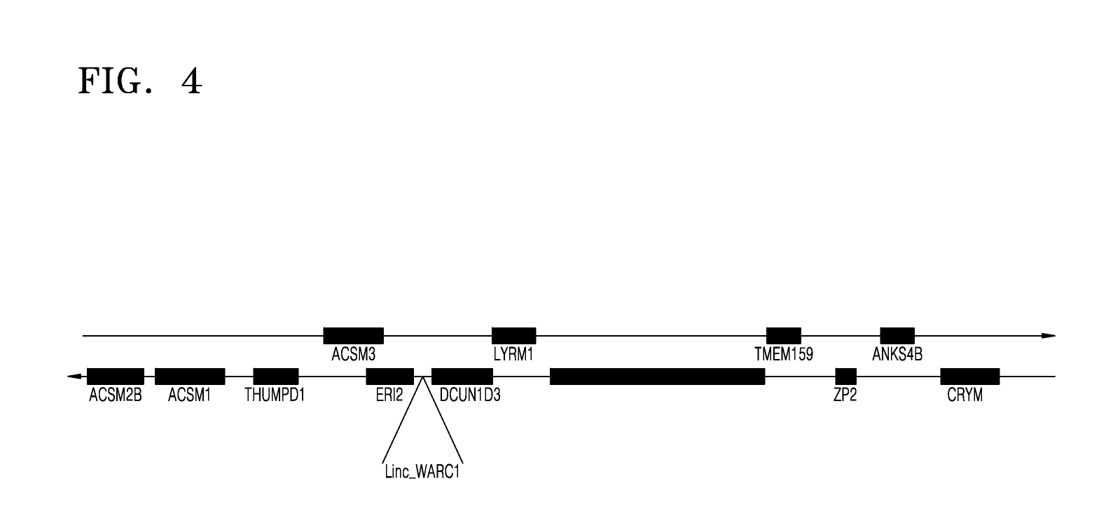

Linc_WARC1 is a nucleic acid having a nucleotide sequence of GenBank Accession No. AK027199 and located at position 12.3 on p-arm of human chromosome 16 (Chr16: 20773748-20775847) and is an intergenic Linc RNA located between an ERI2 gene and a DCUN1D3 gene. The location of Linc_WARC1 is shown in FIG. 4.

(6) Identification of Linc_WARC1 in Progeria Cells

It was identified whether expression levels of Linc_WARC1 increased in progeria cells.

Progeria cells obtained from dermal fibroblast cells of a patient with Hutchinson-Gilford Progeria Syndrome (HGPS) (ATCC®, AG03198B) were cultured in a DMEM supplemented with 10 to 15% (v/v) FBS at 37° C. in 5% CO₂ atmosphere according to Example 1-(1) to prepare young cells and senescent cells. Cells (passage number 11) having a cell doubling time of about 5 days were used as the young cells, and cells (passage number 17) having a cell doubling time of about 14 days were used as the senescent cells.

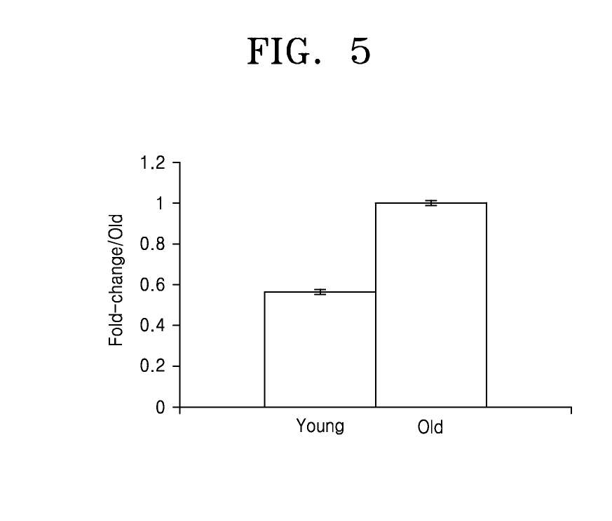

RNAs were obtained from the cultured cells according to the method described above with reference to Example 1-(1), and RT-qPCR was performed according to the method described above with reference to Example 1-(2). The amounts of the quantified transcripts were normalized with respect to an amount of GAPDH, and fold-changes with respect to the transcripts of the senescent cells (fold-change/old) were calculated. The results are shown in FIG. 5 (Young: young cell and Old: senescent cell).

As illustrated in FIG. 5, the expression levels of Linc_WARC1 increased in progeria cells as culture time increases. Thus, it was confirmed that Linc_WARC1 may be used as a biomarker for diagnosing progeria.

It should be understood that the exemplary embodiments described therein should be considered in a descriptive sense only and not for purposes of limitation. Descriptions of features or aspects within each exemplary embodiment should typically be considered as available for other similar features or aspects in other exemplary embodiments.

While one or more exemplary embodiments have been described with reference to the figures, it will be understood by those of ordinary skill in the art that various changes in form and details may be made therein without departing from the spirit and scope as defined by the following claims.

It should be understood that the exemplary embodiments described herein should be considered in a descriptive sense only and not for purposes of limitation. Descriptions of features or aspects within each embodiment should typically be considered as available for other similar features or aspects in other embodiments.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and “at least one” and similar referents in the context of describing the invention (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

What is claimed is:

1. A method of diagnosing a senescence level of a cell or a subject, the method comprising:
separating ribonucleic acids (RNA) from a biological sample of a cell or a subject;
generating complementary DNAs (cDNA) from the separated RNAs;
measuring the amount of SEQ ID NO: 1 or fragment thereof comprising about 4 consecutive nucleotides to about 100 consecutive nucleotides, that is present in the cDNAs; and
comparing the measured amount of SEQ ID NO: 1 or the fragment thereof with the amount of SEQ ID NO: 1 or the fragment thereof obtained from a normal control group sample to determine a senescence level of the cell or the subject.

2. The method of claim 1, wherein the determination of the senescence level is used to diagnose a senescence-associated disease.

3. The method of claim 2, wherein the senescence-associated disease is selected from the group consisting of progeria, cognitive disease, stroke, diabetes, arthritis, arteriosclerosis, heart disease, hair loss, wrinkles, and osteoporosis.

4. The method of claim 3, wherein the senescence-associated disease is a cognitive disease, and the cognitive disease is Alzheimer's disease, Parkinson's disease, dementia, or a combination thereof.

5. The method of claim 1, wherein the determination of senescence level is used to determine the biological age of the subject.

6. The method of claim 1, wherein the biological sample is a sample from a subject having a senescence-associated disease or a risk of having a senescence-associated disease, or a cultured cell.

7. The method of claim 1, wherein measuring the amount of SEQ ID NO: 1 or a fragment thereof is performed by reverse transcription-polymerase chain reaction (RT-PCR), microarray analysis, serial analysis of gene expression (SAGE), Northern blotting, or a combination thereof.

8. The method of claim 1, further comprising determining that the biological sample or subject has an increased senescence level when an amount of SEQ ID NO: 1 or a fragment thereof is about 1.5 times to about 10 times that of the normal control group.

9. The method of claim 1, further comprising determining that the biological sample is a sample having a decreased senescence level when an amount of SEQ ID NO: 1 or a fragment thereof is about 0.1 times to about 0.9 times that of the normal control group.

10. The method of claim 8, wherein the normal control group is a sample separated from a subject or a cell that does not have a senescence-associated disease or a risk of having a senescence-associated disease.

11. The method of claim 9, wherein the normal control group is a sample separated from a subject or a cell that does not have a senescence-associated disease or a risk of having a senescence-associated disease.

12. A method of screening for a senescence inhibitor, the method comprising:
incubating cells with a test compound;
separating RNAs from the cells;
generating cDNAs from the separated RNAs;
measuring the amount of SEQ ID NO: 1 or fragment thereof comprising about 4 consecutive nucleotides to about 100 consecutive nucleotides that is present in the cDNAs; and
determining that the test compound is a senescence inhibitor when an amount of the SEQ ID NO: 1 or the fragment thereof in the cells incubated with the test compound decreases in comparison with a negative control group.


SEQUENCE LISTING (5 sequences)

SEQ ID NO: 1 - DNA, 2118 bp, Artificial Sequence
  Synthetic cDNA complementary to Human Linc_WARC1
cttaatgagtgagcagtaagtctgtgtaagaggctgaatgcatgcccctcagataagccagtacactccttgcttagcaacagaacatcagggtgatgtggagaggggcaggatgtggacgccacattggaaatcggcaacatctgagggcaacaacaaacaagtgtgttgggaaataagaaataactcagttttgacaactggctttgtcagcttttgtgatgtttctttagcagtttattggaaagatggtatgagatgacgtgctgcttcattgaattgctctttcccccatctttgccaaatctcaatgtatcgttcttaaccccacctcctgtaaggggctttgctatgcttcagctggttgtctcagcagctgaagtgctgcccacctgtgtgagttgggtccaggaaaccatgtctgcccttctgataagggaagatgaatctagagctgggtgaagatctaaattttaaccaaacccctgggcccaggaaaataacaattgaaaatgtacaaggcagtgttttcaatattaaacttccccaaggaaagcacaaactagtctttttggaaagggagaaaggattaagccacacagtattagtctttgaagcagtactggtctctaggggctggtgccaaaatggagtcccatagtagttacactcgatggcctcatgtactatatactgtgccgaattgtattaaacagtggtggggagttactgggataagaacttgtctaaaagtttacaaaccaaaacagatctgttaggttggtgcaaaagtaagtttttgccatacttaatgtattgccattaagtatggcaaaaaccacaattacttttacaccaacctatgtatttaagaatgtttggtttgccagattccaaatgaggtcttcagtgcagcaaagcccaaaaggtgtagactcagttatgcaattataaggttaaggcgtagaagaaagctgctgctaggtttttgttgcattttacttgactgctctgctgtttttcttgtctctcatgtttggttagctatgacttgagcatcttggtaactgacaaaggtcttccttgggggacttgaacatcttggtaaatgacaggtcttcttggaggactccagcagtatcttgtttaaacgactgaaaggactattaaggttgttgaattgtgttaattggaactcattgaggaaatgcgacattgatcctcctcttattccacagtgtgttttctgatcatataaagaaggttccgaaccatccatccccctcagagtttattcccctggtaagctgtaattgcatatccagtttaaactggactgggactgcatgttggtgaggatcggcaggggttttcccccttttcgaaagatgaaatagattcttgagcactggttgcagaagccaaaatagttcaaatagctttgcataaccattgggttctgcttctgattcaggtgctgggcatcatgtcctccctattcttctcttcttggaaacccagcctatctcataaatacctacttccggccacccatccaacctccctgctcctttcaacacaaatcttggatattgccaaaggaagccattcagcagctgctggggtttttcatccccctgacatgcatacatttgctctgggagaagtgtcttccctctgaccctggtccccagctccgtctgtgcttaattgctcttacctgttgcactaatgaatatgactaggttttaaagggggaatgtgaagcaataggcacatggggcttggatgaattggtcccacagatataccctgccttaagccgctgaggtgatgagtccactgctcatgtgaccctccacctttgtggatccctcttggtttgtgaccagtgtgtctgtttgttgaggttgtacaaacttgacaaaagttaatacttttgtttgtattttctgcactgttgcactctccaaatggccccttgagtatttttattgacttgttacacacatttttgtctttgatgtctacattttttcctttaatgttttttatttggaaggttacctgctgttggatttaataaatttgtttacttgaaaaaaaaaaaaaaaaaaa

SEQ ID NO: 2 - DNA, 20 bp, Artificial Sequence
  Synthetic Forward primer for human Linc_WARC1
ttaagcacagacggagctgg

SEQ ID NO: 3 - DNA, 20 bp, Artificial Sequence
  Synthetic Backward primer for human Linc_WARC1
tgggcatcatgtcctcccta

SEQ ID NO: 4 - DNA, 20 bp, Artificial Sequence
  Synthetic Forward primer for human Linc_WARC1
cacaaaggtggagggtcaca

SEQ ID NO: 5 - DNA, 20 bp, Artificial Sequence
  Synthetic Backward primer for human Linc_WARC1
tggggtttttcatccccctg